(19) United States
(12) Patent Application Publication
(10) Pub. No.: US 2014/0031649 A1
(43) Pub. Date: Jan. 30, 2014

(54) MOLECULAR CONCENTRATION MEASUREMENT DEVICE AND MOLECULAR CONCENTRATION MEASUREMENT METHOD

(71) Applicant: Sony Corporation, Tokyo (JP)

(72) Inventor: Isamu Nakao, Tokyo (JP)

(21) Appl. No.: 13/944,551

(22) Filed: Jul. 17, 2013

(30) Foreign Application Priority Data
Jul. 24, 2012 (JP) 2012-163248

(51) Int. Cl. A61B 5/1455; A61B 5/145

(52) U.S. Cl. CPC A61B 5/1455; A61B 5/14532
USPC 600/322

(57) ABSTRACT

Provided is a molecular concentration measurement device including a laser oscillator oscillating and outputting first laser light with an oscillation frequency ω₁ and second laser light with an oscillation frequency ω₂ which are in a relationship of ω_{χ}=ω₁−ω₂ (ω₁>ω₂) with regard to an oscillation frequency ω_{χ} that is a molecule oscillation mode of molecules to be measured, a condensing lens condensing the first laser light and the second laser light into a blood vessel of an organism in which the molecules to be measured are included, a light sensing unit sensing stimulated Raman scattering light emitted by the molecules to be measured when the first laser light and the second laser light are radiated on the molecules to be measured, and then Stokes-shifted, and a concentration computation unit computing a concentration of the molecules to be measured from a spectral intensity of the sensed stimulated Raman scattering light.

### BACKGROUND

The present disclosure relates to a molecular concentration measurement device and a molecular concentration measurement method, and particularly to a molecular concentration measurement device and a molecular concentration measurement method that enable measurement of the concentration of molecules to be measured using a favorable S/N ratio.

For diabetic patients, levels of glucose in blood (blood sugar) are monitored at all times, and insulin or a therapeutic medicine is administered to the patients according to changes of the blood-sugar level. However, in recent years, as a method for measuring the blood-sugar levels, the concentration of glucose included in an interstitial fluid extracted from blood or a dermis of skin is generally obtained using an enzymatic method. For this reason, specimens are collected using syringes, or the like, which accordingly causes patients to be concerned not only for infectious disease but also for pain that may arise therefrom.

The applicant of Japanese Patent No. 4618341 has therefore proposed a non-invasive blood sugar measurement method using coherent anti-Stokes Raman scattering (hereinafter referred to as CARS) therein. In this method, since CARS signals output only from a local part inside a body such as red blood cells can be obtained by optically focusing thereon, the amount of glycosylated hemoglobin can be measured in a non-invasive manner. FIG. 1 shows a CARS signal of an aqueous solution of glucose disclosed in “Glucose Concentration Measured by the Hybrid Coherent Anti-Stokes Raman Scattering Technique” written by Xi Wang, Aihua Zhang, Miaochan Zhi, Alexei V. Sokolov, and George R. Welch, published in Physical Review A, Vol. 81, 013813 (2010).

### SUMMARY

However, in this method, since the real part of the third-order non-linear optical susceptibility has finite values other than 0 even in a range outside of a resonant frequency, noise is also amplified at the same time as signals. As shown in FIG. 1, while the peak of a CARS signal of an aqueous solution of glucose is maximally at about 17000 count, noise is even at 9000 count. Thus, when there are a number of molecules other than molecules to be measure, there are cases in which signals are buried by amplified noise due to the presence of other molecules that have a finite value in the real part of the three-dimensional non-linear susceptibility in the wavelength range of the CARS signal.

FIG. 2 shows measurement results of CARS of glucose included in blood of a pig disclosed in “Glucose Concentration Measured by the Hybrid Coherent Anti-Stokes Raman Scattering Technique written by Xi Wang, Aihua Zhang, Miaochan Zhi, Alexei V. Sokolov, and George R. Welch, disclosed in Physical Review A, Vol. 81, 013813 (2010).”

Referring to FIG. 2, CARS of glucose was measured with respect to a pure blood sample collected from the pig, and samples obtained by adding glucose to the collected blood so that the samples have concentrations of 25 mM, 58 mM, 125 mM, 189 mM, 308 mM, and 415 mM. Among the measurement results arranged in the longitudinal direction of FIG. 2, the lowest measurement result is of the pure blood sample, and the higher the graphs are disposed, the higher the concentrations of added glucose are. It can be found that, as the concentrations of added glucose gradually increase, the intensities of the wavenumber portions indicated by the dashed line gradually increase. Thus, it is found that the portions correspond to CARS signals of glucose, but with regard to the CARS signal of the pure blood sample shown in the lowest part of FIG. 2, the CARS signal of glucose is weak for noise derived from other molecules in the blood.

It is desirable to enable concentration measurement of molecules to be measured using a favorable S/N ratio.

According to an embodiment of the present technology, there is provided a molecular concentration measurement device including a laser oscillator that oscillates and outputs first laser light with an oscillation frequency ω₁ and second laser light with an oscillation frequency ω₂ which are in a relationship of ω_{χ}=ω₁−ω₂ (ω₁>ω₂) with regard to an oscillation frequency ω_{χ} that is a molecule oscillation mode of molecules to be measured, a condensing lens that condenses the first laser light and the second laser light into a blood vessel of an organism in which the molecules to be measured are included, a light sensing unit that senses stimulated Raman scattering light that is emitted by the molecules to be measured when the first laser light and the second laser light are radiated on the molecules to be measured, and then Stokes-shifted, and a concentration computation unit that computes a concentration of the molecules to be measured from a spectral intensity of the sensed stimulated Raman scattering light.

According to an embodiment of the present technology, there is provided a molecular concentrate measurement method of a molecular concentration measurement device including oscillating and outputting, by a laser oscillator, first laser light with an oscillation frequency ω₁ and second laser light with an oscillation frequency ω₂ which are in a relationship of ω_{χ}=ω₁−ω₂ (ω₁>ω₂) with regard to an oscillation frequency ω_{χ} that is a molecule oscillation mode of molecules to be measured, condensing, by a condensing lens, the first laser light and the second laser light into a blood vessel of an organism in which the molecules to be measured are included, sensing, by a light sensing unit, stimulated Raman scattering light that is emitted by the molecules to be measured when the first laser light and the second laser light are radiated on the molecules to be measured, and then Stokes-shifted, and computing, by a concentration computation unit, a concentration of the molecules to be measured from a spectral intensity of the sensed stimulated Raman scattering light.

According to an embodiment of the present technology, the first laser light with the oscillation frequency ω₁ and the second laser light with the oscillation frequency ω₂, which are in the relationship of ω_{χ}=ω₁−ω₂ (ω₁>ω₂) with regard to the oscillation frequency ω_{χ} that is the molecule oscillation mode of the molecules to be measured, are oscillated and output, the first laser light and the second laser light are condensed into a blood vessel of an organism in which the molecules to be measured are included, stimulated Raman scattering light, which is emitted by the molecules to be measured when the first laser light and the second laser light are radiated on the molecules to be measured and then Stokes-shifted, is sensed, and the concentration of the molecules to be measured is computed from the spectral intensity of the sensed stimulated Raman scattering light.

The molecular concentration measurement device may be an independent device, or may be an internal block that is included in one device.

According to the embodiments of the present technology described above, the concentration of molecules to be measured can be measured using a favorable S/N ratio.

### BRIEF DESCRIPTION OF THE DRAWINGS

FIG. 1 is a diagram showing a CARS signal of an aqueous glucose solution;

FIG. 2 is a diagram showing an example of CARS measurement results of glucose;

FIGS. 3A to 3C are diagrams for describing stimulated Raman scattering;

FIG. 4 is a diagram showing the square, the real part, and the imaginary part of the absolute value of a third-order non-linear optical susceptibility χ⁽³⁾;

FIG. 5 is a diagram showing a configuration example of a first embodiment of a molecular concentration measurement device to which the present technology is applied;

FIG. 6 is a diagram showing wavelength dependency of absorptive scattering of a biological tissue;

FIG. 7 is a diagram showing spontaneous Raman spectra of water and aqueous solutions of glucose with the concentrations of 1 g/dL and 50 g/dL;

FIG. 8 is a diagram showing a configuration example of a second embodiment of the molecular concentration measurement device to which the present technology is applied;

FIG. 9 is a graph showing two-dimensional data of stimulated Raman scattering light; and

FIG. 10 is a graph in which a light intensity in the spectral range of the stimulated Raman scattering light is plotted on a time axis.

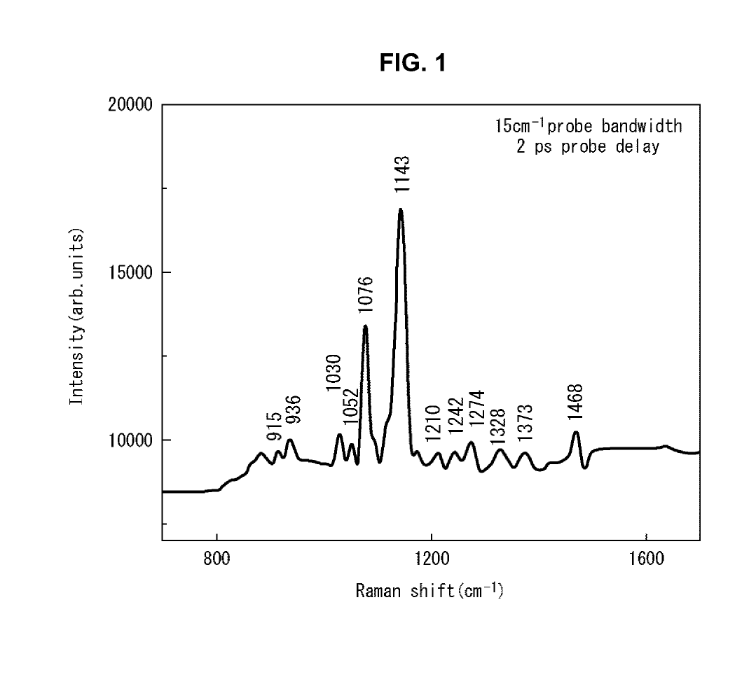

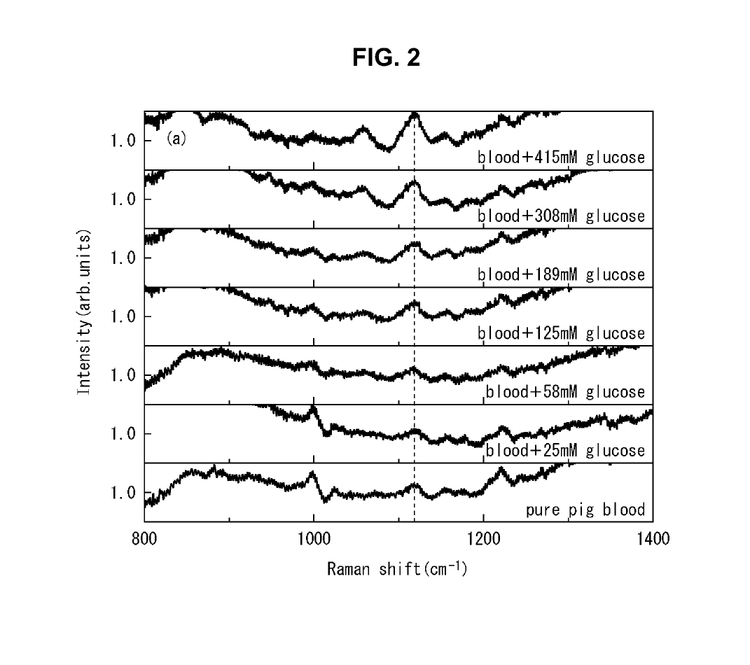

### DETAILED DESCRIPTION OF THE EMBODIMENTS

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

Hereinafter, modes for implementing the present disclosure (hereinafter referred to as embodiments) will be described. It should be noted that description will be provided in the following order.

1. First embodiment (a configuration example in which signal light is detected by a lock-in amplifier)

2. Second embodiment (a configuration example in which signal light is detected by a streak camera)

### 1. First Embodiment

### [Description of Stimulated Raman Scattering]

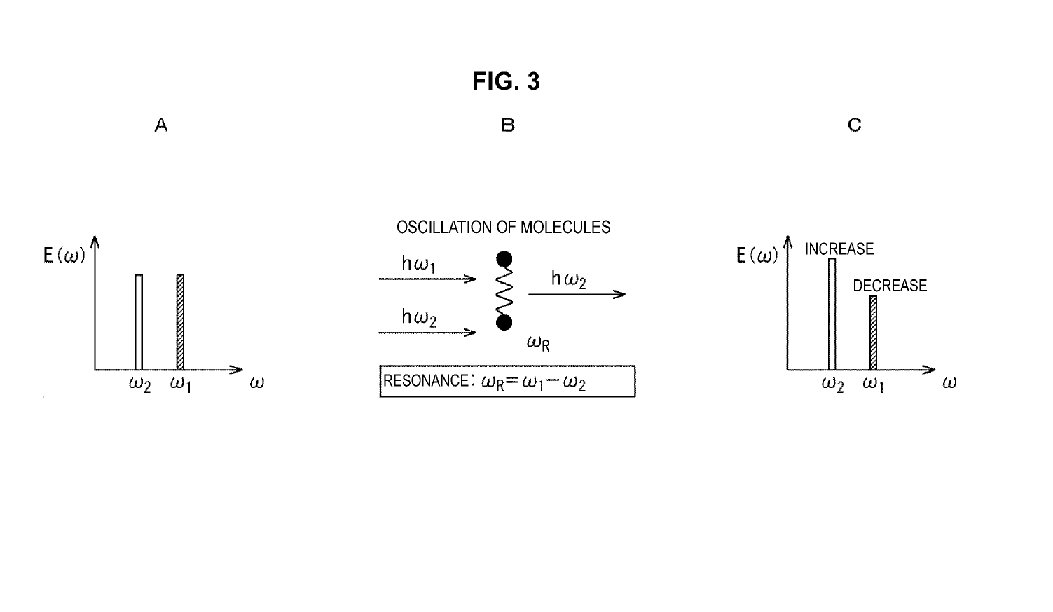

A molecular concentration measurement device to which the present technology is applied measures the concentration (amount) of molecules which are measurement targets (hereinafter, also referred to as molecules to be measured) using stimulated Raman scattering (hereinafter, also referred to as SRS (Stimulated Raman Scattering)). First, stimulated Raman scattering will be described with reference to FIGS. 3A to 3C.

In stimulated Raman scattering, two laser beams with an oscillation frequency ω₁ and an oscillation frequency ω₂ are prepared as shown in FIG. 3A. With regard to ω_{χ} that is a molecular vibration mode of molecules to be measured as shown in FIG. 3B, the oscillation frequencies ω₁ and ω₂ (ω₁>ω₂) of the two laser beams are set to be in the relationship of ω_{χ}=ω₁−ω₂.

When the laser beam with the oscillation frequency ω₁ as excitation light is radiated on the molecules to be measured, the molecules emit spontaneous Raman scattering light with the oscillation frequency ω₂, but in stimulated Raman scattering, the excitation density of the laser beam with the oscillation frequency ω₁ that is excitation light is elevated, and the laser beam with the oscillation frequency ω₂ is radiated on the molecules as seed light (Stokes light), causing Raman scattering light to be stimulated and emitted.

In stimulated Raman scattering, as shown in FIG. 3C, energy moves (Stokes-shifts) to the side of the laser beam with the lower oscillation frequency ω₁ in the two laser beams with the oscillation frequencies ω₁ and ω₂ radiated on the molecules to be measured with the oscillation frequency ω_{χ}, and thereby energy of the laser beam with lower oscillation frequency ω₁ increases. The probability of this stimulated emission phenomenon is proportional to the energy density of incident light.

“The Structure of a Material I—Spectroscopy the First Part” in Vol. 9 of the fifth edition of Experimental Chemistry Course published by Maruzen Co., Ltd., and edited by The Chemical Society of Japan describes on pages 496 and 497 that signal intensities of CARS and SRS are expressed in Equations (1) and (2) as follows.

$$\begin{matrix}{I}_{CARS} = \frac{256 {π}^{4} {ω}_{3}^{2}}{{n}_{1}^{2} {n}_{2} {n}_{3} {c}^{4}} {\vert {χ}^{( 3 )} \vert}^{2} {I}_{1}^{2} {I}_{2} {L}^{2} {\{ sin c \frac{Δ KL}{2} \}}^{2} & ( 1 ) \\ \frac{Δ {I}_{1}}{{I}_{1}} = - \frac{32 {π}^{2} {ω}_{1}}{{n}_{1} {n}_{2} {c}^{2}} {I}_{m} {χ}^{( 3 )} {I}_{2} L & ( 2 )\end{matrix}$$

Here, n₁, n₂, and n₃ respectively indicate refractive indexes of a sample in the oscillation frequencies (frequencies) ω₁, ω₂, and ω₃ (=2ω₁−ω₂), χ⁽³⁾ indicates a third-order non-linear optical susceptibility, L indicates the length of the sample, and I₁ and I₂ respectively indicate the intensities of excitation light and Stokes light. ΔK is expressed as ΔK=2k₁−k₂−k₃ by setting wavenumber vectors of the frequencies ω₁, ω₂, and ω₃ to be k₁, k₂, and k₃.

A CARS signal is in proportion to the square of the absolute value of the third-order non-linear optical susceptibility χ⁽³⁾ as understood from Equation (1), and a gain of SRS is in proportion to the imaginary part of the third-order non-linear optical susceptibility χ⁽³⁾ as understood from Equation (2).

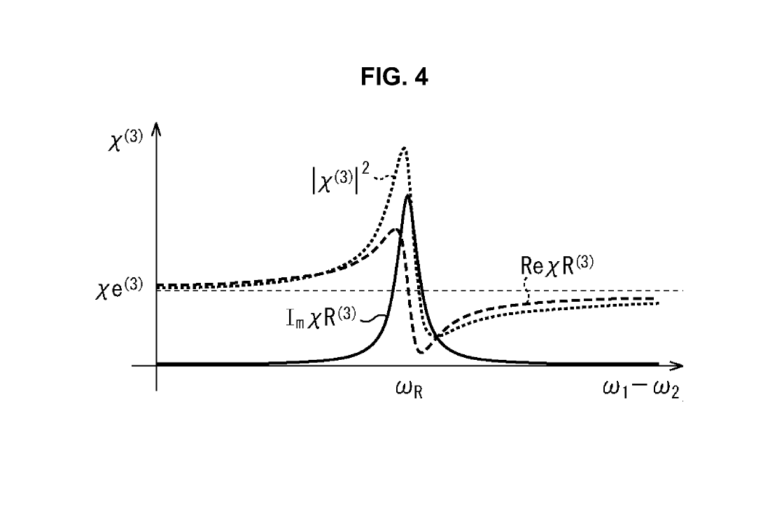

FIG. 4 is a diagram showing the square of the absolute value of the third-order non-linear optical susceptibility χ⁽³⁾, the real part of the third-order non-linear optical susceptibility χ⁽³⁾, and the imaginary part of the third-order non-linear optical susceptibility χ⁽³⁾.

As shown in FIG. 4, while the imaginary part of the third-order non-linear optical susceptibility χ⁽³⁾ has a single peak on a resonance frequency ω_{R}, and has the value of 0 on frequencies other than that, the real part of the third-order non-linear optical susceptibility χ⁽³⁾ describes an S-shaped curve on the resonance frequency ω_{R}, and has finite values other than 0 on frequencies other than that.

Thus, since the CARS signal that is in proportion to the square of the absolute value of the third-order non-linear optical susceptibility χ⁽³⁾ is generated even on frequencies other than the resonance frequency ω_{R}, when there are molecules other than molecules to be measured even though the wavelength is adjusted to the resonance of the molecules to be measured, a CARS signal is generated even from the other molecules. The signal appears as amplified noise.

Meanwhile, as shown by Equation (2), stimulated Raman scattering does not depend on the real part of the third-order non-linear optical susceptibility χ⁽³⁾ that has finite values other than 0 in non-resonance, and generates signals only by the imaginary part having the single peak only on the resonance wavelength. Thus, stimulated Raman scattering is useful when a trace amount of molecules to be measured is measured using a specimen including a variety of kinds of molecules such as an organism.

### [Configuration Example of Molecular Concentration Measurement Device]

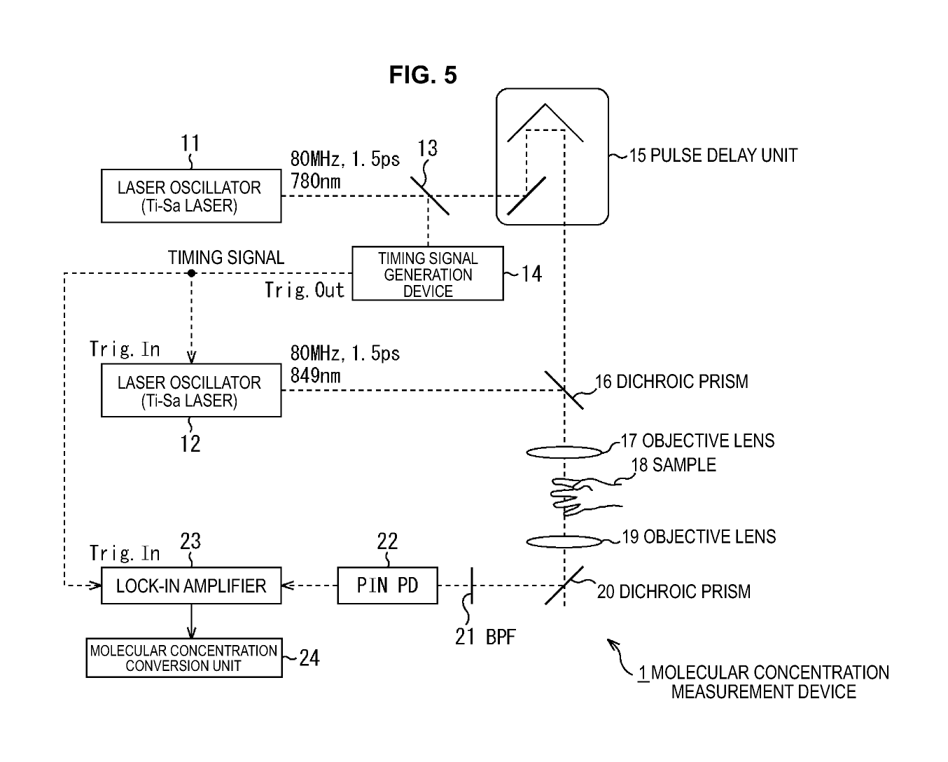

FIG. 5 is a diagram showing a configuration example of a molecular concentration measurement device that measures concentration of molecules to be measured using stimulated Raman scattering.

The molecular concentration measurement device 1 of FIG. 5 is a device for measuring the concentration (amount) of glucose, as the molecules to be measured in a blood vessel of an organism of a diabetic patient.

Laser oscillators 11 and 12 are configured to have, for example, a mode-locked titanium sapphire (Ti-Sa) laser, or the like that oscillates by exciting a crystal of titanium sapphire using a mode synchronizing method, and to output pulse laser light of which a transverse mode is TEM00.

The laser oscillator 11 oscillates laser light as excitation light in stimulated Raman scattering, and outputs laser light with a wavelength λ₁ of, for example, 780 nm as a pulse laser with a pulse width of 1.5 ps [picoseconds] in a pulse cycle of 80 MHz.

The laser oscillator 12 oscillates laser light serving as Stokes light in stimulated Raman scattering, and outputs laser light with a wavelength λ₂ of, for example, 849 nm as a pulse laser with a pulse width of 1.5 ps in a pulse cycle of 80 MHz.

It should be noted that the laser oscillators 11 and 12 may also use not only other mode-locked lasers such as mode-locked fiber lasers and mode-locked semiconductor lasers but also Q-switched lasers, etc.

Pulse laser light of the excitation light output from the laser oscillator 11 is split into two optical paths of a timing signal generation device 14 and a pulse delay unit 15 by a beam splitter 13.

The timing signal generation device 14 generates timing signals used so as to be in frequency synchronization with the pulse frequency (80 MHz) of the laser oscillator 11 based on the pulse laser light output from the laser oscillator 11, and then supplies the signals to the laser oscillator 12 and a lock-in amplifier 23.

The pulse delay unit 15 adjusts pulse timings of excitation light and Stokes light by moving a delay optical system using a piezoelectric element. For example, the pulse delay unit 15 adjusts the deviation between pulse phases of the excitation light and the Stokes light of a sample 18 so as to be shorter than or equal to 100 fs [femtoseconds].

The excitation light of which timing is adjusted by the pulse delay unit 15 and the Stokes light output from the laser oscillator 12 are multiplexed in a dichroic prism 16, and are incident on an objective lens 17 with a numerical aperture of about 0.7 to 1.2. The objective lens 17 condenses the incident excitation light and Stokes light on the sample 18. The sample 18 is a finger or the like of a diabetic patient, and a focus position of laser light condensed by the objective lens 17 is set to be an inner part of the diabetic patient as the sample 18, to be specific, an inner side of a blood vessel with a depth of 1 mm or less from a surface of an organism.

Due to momentary strong excitation by a pulse laser and condensation by the objective lens 17, the excitation light of which the density of excitation has increased is radiated on glucose that is a molecule to be measured in the blood vessel of the sample 18, and accordingly, stimulated Raman scattering light undergoes stimulated emission. By setting the wavelength λ₁ of the excitation light to be 780 nm and the wavelength λ₂ of the Stokes light to be 849 nm, stimulated emission of the spectral peak (wavelength of 849 nm) of the Raman scattering light with a Stokes shift having the wavenumber of 1040 cm⁻¹ can be attained. The wavelength λ₂ of the Stokes light is set to a wavelength within spectral distribution of spontaneous Raman scattering light, for example, so as to be within a wavelength range in which a spectral intensity is equal to or higher than the half value of the spectral peak.

It should be noted that, if the an air layer is set in the space between the sample 18 that is a finger or the like of a diabetic patient and the objective lens 17, the refractive index of the inner side of the sample 18 is different from that of the air layer on the top surface of the sample, and thus scattering of laser light arises on the surface of the sample 18. Therefore, in order to suppress light scattering occurring on the surface of the sample 18, the space between the objective lens 17 and the sample 18 is filled with a liquid such as water or oil, or gel, and the sample 18 and the surface thereof can thereby be set to have the same refractive index.

Stimulated Raman scattering light that has penetrated through or been reflected by the sample 18 is condensed on another objective lens 19, and then guided to another dichroic prism 20. Since light incident on the dichroic prism 20 includes laser light with the wavelength λ₁ of 780 nm in addition to laser light with the wavelength λ₂ of 849 nm that is stimulated Raman scattering light, the dichroic prism 20 separates the incident light into the laser light with the wavelength λ₂ and the laser light with the wavelength λ₁. To be specific, the dichroic prism 20 reflects the laser light with the wavelength λ₂ that is the stimulated Raman scattering light so as to be incident on a BPF (Band-Pass Filter) 21, and causes the laser light with the wavelength λ₁ to penetrate therethrough.

The BPF 21 is a filter through which the laser light with the wavelength λ₂ of 849 nm that is the stimulated Raman scattering light passes, further enhancing selectability of the laser light of 849 nm. A PIN PD (Photodiode) 22 senses the laser light that has passed through the BPF 21 and then performs photoelectric conversion thereon.

The lock-in amplifier 23 performs lock-in detection on electric signals output by the PIN PD 22 using timing signals supplied from the timing signal generation device 14 as a reference signal. The lock-in amplifier 23 converts the electric signals output by the PIN PD 22 into voltage values and then outputs the values to a molecular concentration conversion unit 24.

It should be noted that the sensitivity of the detection can be further enhanced not only by performing lock-in detection in accordance with the cycle of a laser pulse but also by performing lock-in detection in accordance with a cycle of external intensity modulation in such a way that an external intensity modulator (not shown) is installed near the dichroic prism 16 on any side of the laser light with the wavelength λ₁ and the laser light with the wavelength of λ₂, and the constant intensity of the laser pulse of the laser light with the wavelength λ₁ or the wavelength λ2 is subjected to sinusoidal intensity-modulation in a longer cycle (for example, 40 MHz) than the cycle of the pulse using the external intensity modulator (Reference document: OPTICS EXPRESS, Vol. 18, No. 13, 13708, written by Yasuyuki Ozeki, Yuma Kitagawa, Kazuhiko Sumimura, Norihiko Nishizawa, Wataru Umemura, Shin'ichiro Kajiyama, Kiichi Fukui, and Kazuyoshi Itoh).

The molecular concentration conversion unit 24 computes the spectral intensity of stimulated Raman scattering light from the voltage values supplied from the lock-in amplifier 23, and further computes the concentration (amount) of glucose in the blood vessel of the diabetic patient that is the sample 18 based on the spectral intensity of the stimulated Raman scattering light.

The flow of measurement performed by the molecular concentration measurement device 1 configured as described above is as follows. First, a finger or the like of a diabetic patient (organism) is placed in a predetermined position near the objective lens 17 as the sample 18. Next, a pulse laser that is excitation light is output from the laser oscillator 11, and a pulse laser that is Stokes light is output from the laser oscillator 12.

The pulse laser beams of the excitation light and the pulse laser of the Stokes light are condensed by the objective lens 17, and then radiated on glucose that is a molecule to be measured in a blood vessel of the diabetic patient as the sample 18.

Since the excitation light radiated on the glucose serving as a molecule to be measured has increased excitation density due to momentary strong excitation by a pulse laser and condensation by the objective lens 17, stimulated Raman scattering light undergoes stimulated emission. As described above, when the wavelength λ₁ of the excitation light is set to be 780 nm and the wavelength λ₂ of Stokes light is set to be 849 nm, stimulated emission of the spectral peak (wavelength of 849 nm) of the Raman scattering light with a Stokes shift having the wavenumber of 1040 cm⁻¹ is attained.

The stimulated Raman scattering light that has undergone stimulated emission is sensed by the PIN PD 22 via the dichroic prism 20 and the BPF 21. The stimulated Raman scattering light sensed by the PIN PD 22 is converted into a voltage value by the lock-in amplifier 23. Then, the molecular concentration conversion unit 24 converts the voltage value into the intensity of the stimulated Raman scattering light, and further computes the concentration (amount) of glucose serving as the molecule to be measured from the intensity of the stimulated Raman scattering light.

In the measurement of the molecular concentration measurement device 1 described above, a single spectral peak of Raman scattering light (spectral peak with the wavenumber of 1040 cm⁻¹) is detected, but it may be possible to detect a plurality of spectral peaks. In such a case, the molecular concentration measurement device 1 can detect the intensity of Raman scattering light of a spectral peak with a wavenumber different from a first spectral peak by changing the wavelength of a pulse laser from at least one of the laser oscillator 11 and the laser oscillator 12 after the first spectral peak is detected as described above. In addition, when a mode-locked semiconductor laser, or the like that is more inexpensive than a mode-locked titanium sapphire laser is used for the laser oscillators 11 and 12, a plurality of laser oscillators that output pulse lasers having different wavelengths may be prepared for excitation light and Stokes light so as to switch a laser oscillator that outputs pulse lasers having a first spectral peak and a second spectral peak.

In addition, in the configuration of the molecular concentration measurement device 1 described above, two laser oscillators 11 and 12 are provided so that laser light beams with the wavelength λ₁ for excitation light and the wavelength λ₂ for Stokes light are output from different laser oscillators. However, only one laser oscillator may be configured so that laser light beams with two different wavelengths λ₁ and λ₂ are generated from laser light with a predetermined wavelength output from the laser oscillator. In other words, the functions of the two laser oscillators 11 and 12 may be realized by one laser oscillator.

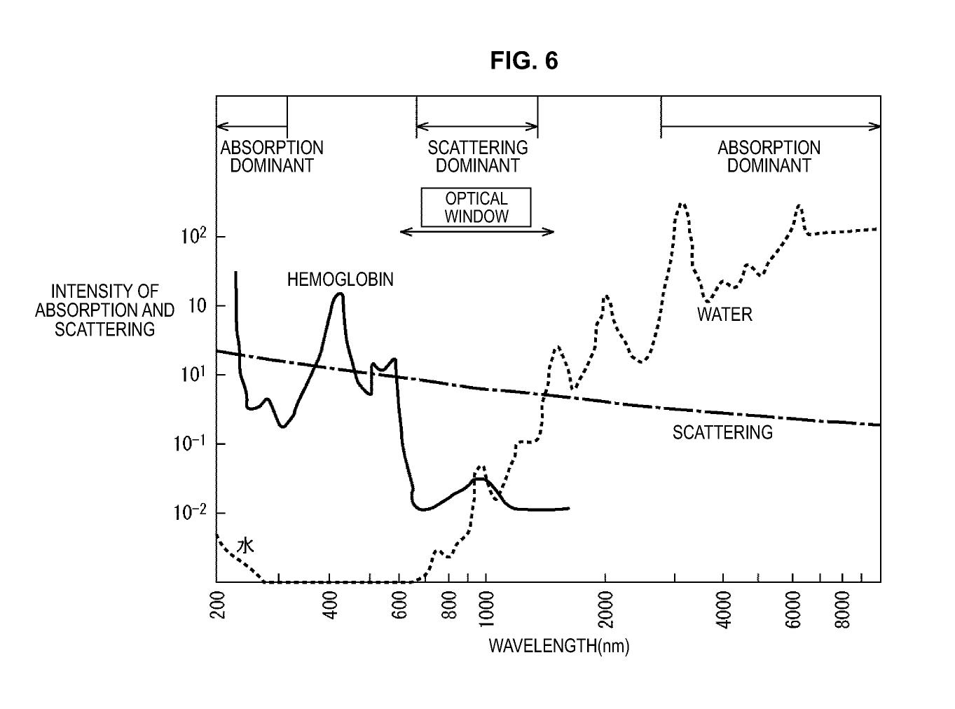

In the measurement described above, the wavelength λ₁ of excitation light is set to be 780 nm, and the wavelength λ₂ of Stokes light is set to be 849 nm, but the wavelength of laser light may be set within the range from 700 nm to 2 μm. FIG. 6 shows wavelength dependency of absorptive scattering of a biological tissue, and since optical absorption is low in the range from 700 nm to 2 μm that is also called an optical window of an organism, as shown in FIG. 6, laser light easily permeates a biological tissue. For this reason, laser light can be efficiently radiated on molecules to be measured by setting the wavelength of the laser light to be within the range from 700 nm to 2 μm.

It should be noted that, in the measurement described above, the pulse widths of the excitation light and Stokes light are set to be 1.5 ps, and the deviation between the pulse timings (pulse phases) of the excitation light and Stokes light is adjusted to be shorter than or equal to 100 fs. An overlap of the pulses of the excitation light and Stokes light in this case is 1.4 ps.

However, the pulse widths of the excitation light and Stokes light may be equal to or longer than 100 fs for the following reason.

According to Heisenberg's uncertainty principle, the following relationship as in Equation (3) is elicited from the uncertainty of energy ΔE and uncertainty of time Δt using Planck's constant h.

$$Δ E \cdot Δ t > \frac{1}{2} \frac{h}{2 π} \qquad ( 3 )$$

Equation (3) shows the relationship between broadening of time of a laser pulse and broadening of energy, in other words, the relationship between broadening of time of a laser pulse and spectral broadening of a laser.

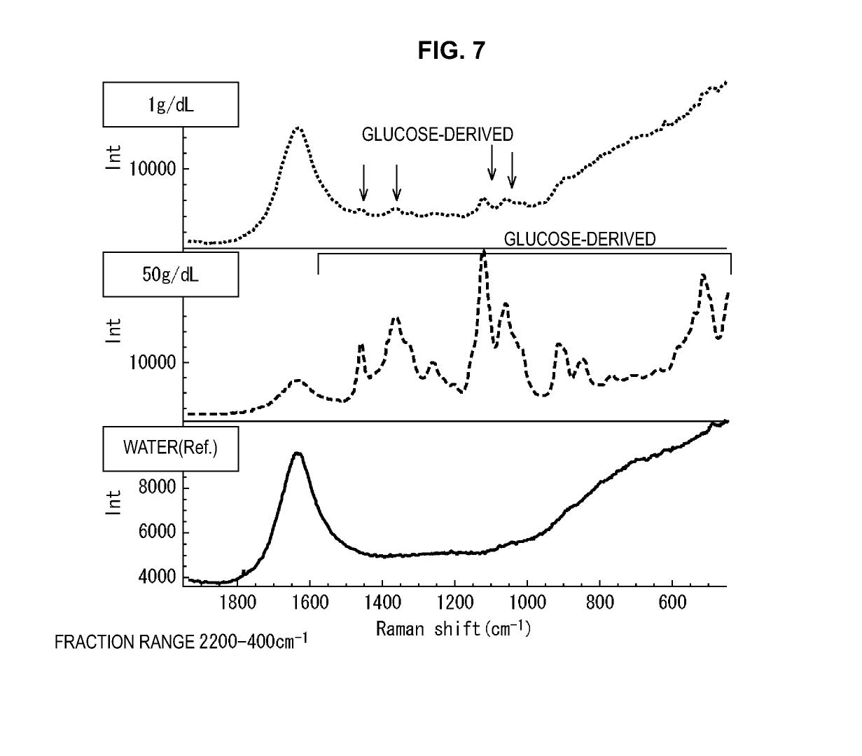

Meanwhile, a peak of a spontaneous Raman spectrum generally has a spectral width of about 10 to 20 cm⁻¹. FIG. 7 shows spontaneous Raman scattering spectra of aqueous solutions of glucose with concentrations of 1 g/dL and 50 g/dL and water as examples of spontaneous Raman spectra.

It is found that, even in the spectra of the stimulated Raman scattering, peaks appear in the same wavenumber as spontaneous Raman scattering. For this reason, wavenumber resolution of 5 to 10 cm⁻¹ or higher necessary for sampling of a spectral signal is necessary as performance in measurement of stimulated Raman scattering in consideration of a Nyquist theorem. Thus, laser light also has to have the same degree of spectral broadening. When distribution of a wavenumber of 10 cm⁻¹ is converted into energy, the result is 1.24 meV, but when broadening of time of a pulse, i.e., the pulse width, is obtained using Equation (3) based on the value, the result can be computed to be 265 fs or higher. There is also a case considered in which the spectrum of Raman scattering is further broadened than in the above case, but in such a case, the pulse width can also be shortened so as to be about 100 fs based on the same discussion point. Accordingly, in order to obtain sufficient wavenumber resolution in measurement of molecules, the time width of a laser pulse has to be 100 fs or greater. It should be noted that the upper limit of the time width of a laser pulse is, for example, 100 ps.

The laser pulses of excitation light and Stokes light necessarily overlap in the entire period of 100 fs when the pulse width is 100 fs. In addition, when any one pulse width of excitation light and Stokes light has 100 fs, the period in which the pulse width is 100 fs necessarily overlaps the other pulse width. In other words, it is necessary for excitation light and Stokes light to be simultaneously radiated on molecules to be measured at least for the time of 100 fs.

### 2. Second Embodiment

Generally, when SRS or CARS measurement is performed, it is necessary to set a momentary strong excitation state (to input a number of photons of specific energy) in order to cause a non-linear phenomenon. For this reason, the molecular concentration measurement device 1 momentarily and repeatedly irradiates a measurement target with energy pulse-compressed using a mode-locked laser or Q-switched laser. In the case of the mode-locked laser, a pulse rate changes according to the length of a laser oscillator, but in general, pulse laser light of the pulse rate of dozens MHz to several GHz and 1 ps or so can be emitted. In addition, it is found that a time for which a molecule to be measured emits Raman scattering light is about 10 ps. For example, when a laser with a pulse rate of 100 MHz and 1 ps is radiated, the time in which signals are received in the cycle of 10 ns is 10 ps, and when signals are continuously received, no signals are input for 99.9% of the time, and noise is continuously detected for the time. Thus, an S/N ratio can be further enhanced by measuring only the time for which SRS signals are input in an optical receiver (performing gate detection).

### [Configuration Example of Molecular Concentration Measurement Device]

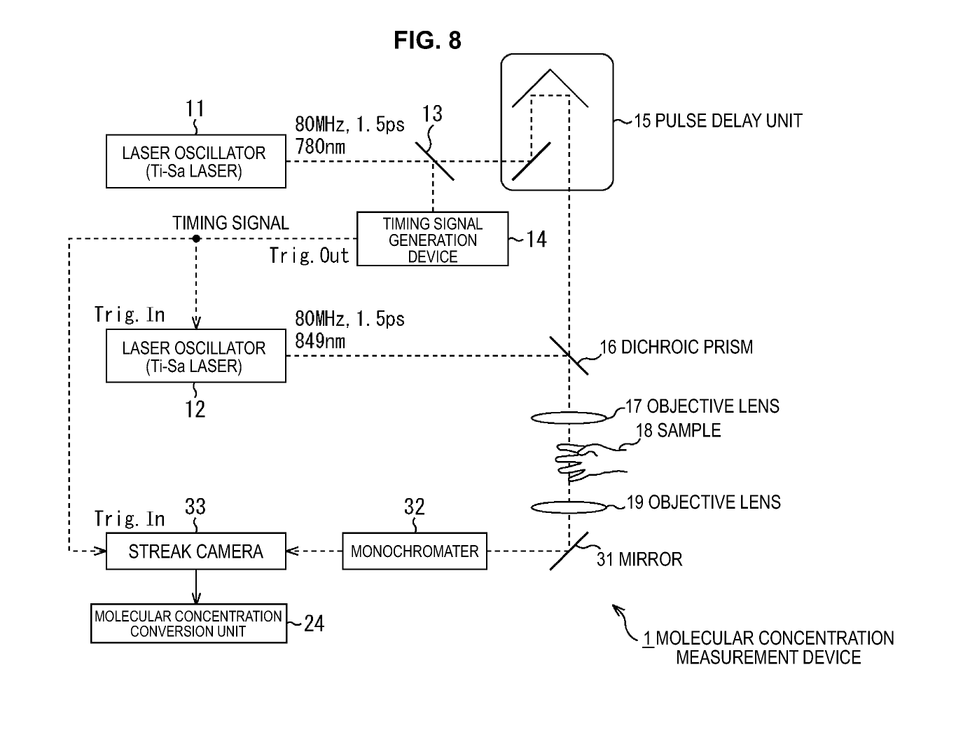

FIG. 8 is a diagram showing a configuration example of a second embodiment of the molecular concentration measurement device, showing a configuration example of the molecular concentration measurement device 1 that performs gate detection on Raman scattering light.

In FIG. 8, the same reference numerals are given to portions corresponding to those of FIG. 5, and description thereof will be appropriately omitted. In the molecular concentration measurement device 1 of FIG. 8, a mirror 31, a monochromator (spectroscope) 32, and a streak camera 33 are provided instead of the dichroic prism 20, the BPF 21, the PIN PD 22, and the lock-in amplifier 23 of FIG. 5.

Stimulated Raman scattering light condensed by an objective lens 19 is reflected on the minor 31 and then guided to the monochromator 32. The monochromator 32 disperses laser light with the wavelength λ₂ that is the stimulated Raman scattering light, and then outputs the light to the streak camera 33.

The streak camera 33 performs repetition time sweeping in the measurement range of 20 ps in a direction perpendicular to the dispersing direction of the monochromator 32 based on a timing signal supplied from the timing signal generation device 14 so as to sense laser light (signal) from the monochromator 32. The length of the optical path is adjusted so that the timing of the time sweeping is delayed by a predetermined time from the incident stimulated Raman scattering light.

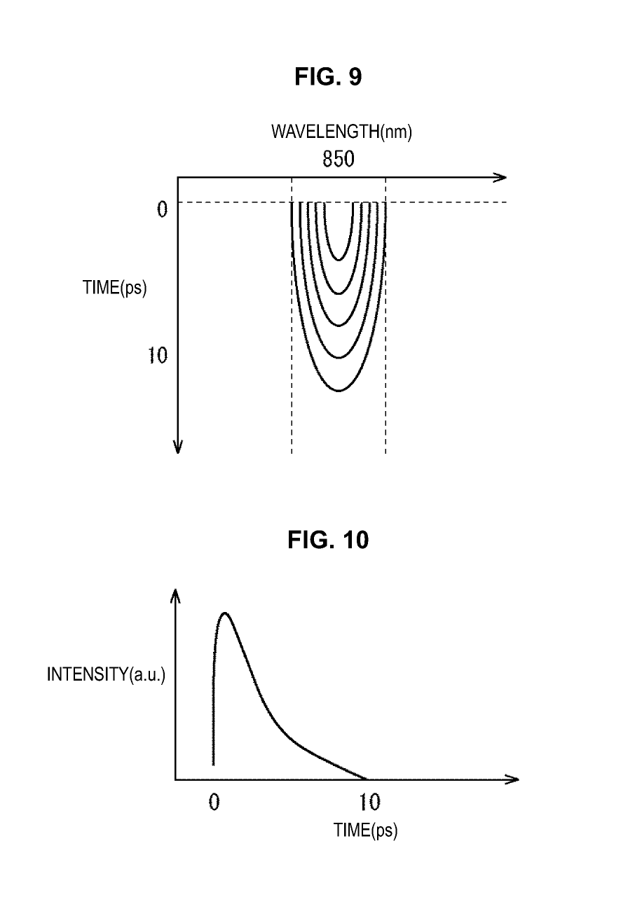

FIG. 9 shows two-dimensional data of stimulated Raman scattering light sensed by a CCD sensor in the streak camera 33. The contour lines of FIG. 9 indicate intensities of signal light.

FIG. 10 is a graph in which a light intensity of the spectral range of the stimulated Raman scattering light (the range narrowed by the vertically dashed lines of FIG. 9) in the two-dimensional data shown in FIG. 9 is plotted on a time axis.

As shown in FIG. 10, the molecular concentration conversion unit 24 integrates the light intensity in the time range from 0 ps to 10 ps in which a signal appears and then disappears so as to compute the spectral intensity of the stimulated Raman scattering light. Further, the molecular concentration conversion unit 24 computes the concentration (amount) of glucose in a blood vessel of a diabetic patient that is the sample 18 based on the spectral intensity of the stimulated Raman scattering light.

In the configuration of the molecular concentration measurement device 1 of FIG. 8, molecules to be measured receive only signals of 10 ps or shorter (relaxation components) after they emit stimulated Raman scattering light, and then the spectral intensity of the stimulated Raman scattering light is computed. Accordingly, since only times in which signal light corresponding to the stimulated Raman scattering light is input to the streak camera 33 can be measured, noise can be drastically reduced, and the S/N ratio can thereby be enhanced.

It should be noted that, as understood with reference to FIG. 7, a Raman spectrum derived from water is expressed near the intensity of 1650 cm⁻¹, and the peak intensity of the Raman spectrum is approximately proportional to the number of water molecules. It can be said that the same applies even to the peak intensity of glucose. Thus, the molecular concentration conversion unit 24 can create and store in advance respective calibration curves indicating the relationship between the peak intensity of the Raman spectrum and the number of molecules for water and glucose, obtain the number of molecules of each of the water and glucose from the measured peak intensity of the Raman spectrum so as to acquire the ratio of the molecules, and thereby obtain the concentration of glucose with respect to water.

As described above, the molecular concentration measurement device 1 to which the present technology is applied can measure the level of blood sugar (glucose) without collecting blood or a body fluid from patients. In other words, since the device can measure the level of blood sugar in a non-invasive manner, infectious disease can be prevented without causing pain to patients.

Since the molecular concentration measurement device 1 can measure the level on a surface of an organism as well as the level in a deep part 1 mm or less away from the surface, the level of glucose in a blood vessel and other amounts of biomolecules can be measured.

In addition, since the molecular concentration measurement device 1 measures the concentration of molecules to be measured using stimulated Raman scattering, concentration of the molecules to be measured can be measured using a favorable S/N ratio.

Embodiments of the present technology are not limited to the above-described embodiments, and can be variously modified within the scope not departing from the gist of the present technology.

It should be understood by those skilled in the art that various modifications, combinations, sub-combinations and alterations may occur depending on design requirements and other factors insofar as they are within the scope of the appended claims or the equivalents thereof.

The present technology may also be configured as below.
1. A molecular concentration measurement device including:

a laser oscillator that oscillates and outputs first laser light with an oscillation frequency ω₁ and second laser light with an oscillation frequency ω₂ which are in a relationship of ω_{χ}=ω₁−ω₂ (ω₁>ω₂) with regard to an oscillation frequency ω_{χ} that is a molecule oscillation mode of molecules to be measured;

a condensing lens that condenses the first laser light and the second laser light into a blood vessel of an organism in which the molecules to be measured are included;

a light sensing unit that senses stimulated Raman scattering light that is emitted by the molecules to be measured when the first laser light and the second laser light are radiated on the molecules to be measured, and then Stokes-shifted; and

a concentration computation unit that computes a concentration of the molecules to be measured from a spectral intensity of the sensed stimulated Raman scattering light.
(2) The molecular concentration measurement device according to (1),

wherein the laser oscillator is configured to have a mode-locked laser or Q-switched laser oscillating laser light of which a transverse mode is TEM00, and

wherein the condensing lens condenses light in a manner that a position of focus is set to be an inner part of the blood vessel of the organism in which the molecules to be measured are included.
(3) The molecular concentration measurement device according to (1) or (2), wherein a wavelength of the second laser light is set to be a wavelength in spectral distribution of spontaneous Raman scattering light.
(4) The molecular concentration measurement device according to any one of (1) to (3),

wherein the laser oscillator outputs the first laser light and the second laser light as a pulse laser having a pulse width of 100 fs or greater,

wherein the device further includes:
an adjustment unit that adjusts pulse timings of the first laser light and the second laser light in a manner that the first laser light and the second laser light are simultaneously radiated on the molecules to be measured at least for 100 fs.
(5) The molecular concentration measurement device according to any one of (1) to (4),

wherein the light sensing unit senses only a relaxation component of 10 ps or shorter after the molecules to be measured emit the stimulated Raman scattering light.
(6) The molecular concentration measurement device according to any one of (1) to (5),

wherein wavelengths of the first laser light and the second laser light have values within the range from 700 nm to 2 μm.
(7) The molecular concentration measurement device according to any one of (1) to (6),

wherein the concentration computation unit computes the concentration of the molecules to be measured in a manner that the unit stores calibration curves indicating a relationship of a peak intensity of a Raman spectrum and a number of molecules with regard to water and the molecules to be measured in advance, and then obtains a number of molecules of each of the water and the molecules to be measured from the measured peak intensity of the Raman spectrum so as to have the ratio of the number of molecules.
(8) A molecular concentrate measurement method of a molecular concentration measurement device including:

oscillating and outputting, by a laser oscillator, first laser light with an oscillation frequency ω₁ and second laser light with an oscillation frequency ω₂ which are in a relationship of ω_{χ}=ω₁−ω₂ (ω₁>ω₂) with regard to an oscillation frequency ω_{χ} that is a molecule oscillation mode of molecules to be measured;

condensing, by a condensing lens, the first laser light and the second laser light into a blood vessel of an organism in which the molecules to be measured are included;

sensing, by a light sensing unit, stimulated Raman scattering light that is emitted by the molecules to be measured when the first laser light and the second laser light are radiated on the molecules to be measured, and then Stokes-shifted; and

computing, by a concentration computation unit, a concentration of the molecules to be measured from a spectral intensity of the sensed stimulated Raman scattering light.

The present disclosure contains subject matter related to that disclosed in Japanese Priority Patent Application JP 2012-163248 filed in the Japan Patent Office on Jul. 24, 2012, the entire content of which is hereby incorporated by reference.

What is claimed is:

1. A molecular concentration measurement device comprising:
a laser oscillator that oscillates and outputs first laser light with an oscillation frequency ω₁ and second laser light with an oscillation frequency ω₂ which are in a relationship of ω_{χ}=ω₁−ω₂ (ω₁>ω₂) with regard to an oscillation frequency ω_{χ} that is a molecule oscillation mode of molecules to be measured;
a condensing lens that condenses the first laser light and the second laser light into a blood vessel of an organism in which the molecules to be measured are included;
a light sensing unit that senses stimulated Raman scattering light that is emitted by the molecules to be measured when the first laser light and the second laser light are radiated on the molecules to be measured, and then Stokes-shifted; and
a concentration computation unit that computes a concentration of the molecules to be measured from a spectral intensity of the sensed stimulated Raman scattering light.

2. The molecular concentration measurement device according to claim 1,
wherein the laser oscillator is configured to have a mode-locked laser or Q-switched laser oscillating laser light of which a transverse mode is TEM00, and
wherein the condensing lens condenses light in a manner that a position of focus is set to be an inner part of the blood vessel of the organism in which the molecules to be measured are included.

3. The molecular concentration measurement device according to claim 1, wherein a wavelength of the second laser light is set to be a wavelength in spectral distribution of spontaneous Raman scattering light.

4. The molecular concentration measurement device according to claim 1,
wherein the laser oscillator outputs the first laser light and the second laser light as a pulse laser having a pulse width of 100 fs or greater,
wherein the device further comprises:
an adjustment unit that adjusts pulse timings of the first laser light and the second laser light in a manner that the first laser light and the second laser light are simultaneously radiated on the molecules to be measured at least for 100 fs.

5. The molecular concentration measurement device according to claim 1,
wherein the light sensing unit senses only a relaxation component of 10 ps or shorter after the molecules to be measured emit the stimulated Raman scattering light.

6. The molecular concentration measurement device according to claim 1,
wherein wavelengths of the first laser light and the second laser light have values within the range from 700 nm to 2 μm.

7. The molecular concentration measurement device according to claim 1,
wherein the concentration computation unit computes the concentration of the molecules to be measured in a manner that the unit stores calibration curves indicating a relationship of a peak intensity of a Raman spectrum and a number of molecules with regard to water and the molecules to be measured in advance, and then obtains a number of molecules of each of the water and the molecules to be measured from the measured peak intensity of the Raman spectrum so as to have the ratio of the number of molecules.

8. A molecular concentrate measurement method of a molecular concentration measurement device comprising:
oscillating and outputting, by a laser oscillator, first laser light with an oscillation frequency ω₁ and second laser light with an oscillation frequency ω₂ which are in a relationship of ω_{χ}=ω₁−ω₂ (ω₁>ω₂) with regard to an oscillation frequency ω_{χ} that is a molecule oscillation mode of molecules to be measured;
condensing, by a condensing lens, the first laser light and the second laser light into a blood vessel of an organism in which the molecules to be measured are included;
sensing, by a light sensing unit, stimulated Raman scattering light that is emitted by the molecules to be measured when the first laser light and the second laser light are radiated on the molecules to be measured, and then Stokes-shifted; and
computing, by a concentration computation unit, a concentration of the molecules to be measured from a spectral intensity of the sensed stimulated Raman scattering light.
